(19) United States
(12) Patent Application Publication
(10) Pub. No.: US 2023/0035611 A1
(43) Pub. Date: Feb. 2, 2023

(54) INVERSE LATEX FOR A COSMETIC COMPOSITION, COMBINING TETRASODIUM SALT OF GLUTAMIC ACID, N,N-DIACETIC ACID AS A CHELATING AGENT AND A POLYELECTROLYTE COMPRISING AMPS AND ACRYLAMIDE

(71) Applicant: SOCIETE D'EXPLOITATION DE PRODUITS POUR LES INDUSTRIES CHIMIQUES - SEPPIC, Paris (FR)

(72) Inventors: Miruna BODOC, Castres (FR); Aurélie COLAS, La Garenne Colombes (FR)

(21) Appl. No.: 17/783,121

(22) PCT Filed: Dec. 7, 2020
(86) PCT No.: PCT/EP2020/084830

§ 371 Date: Jun. 7, 2022

(30) Foreign Application Priority Data
Dec. 9, 2019 (FR) 1913979

(51) Int. Cl. A61K 8/81; A61K 8/06; A61K 8/44

(52) U.S. Cl. CPC A61K 8/8158; A61K 8/8147; A61K 8/06; A61K 8/44

(57) ABSTRACT

Disclosed is a self-invertible inverse latex including an aqueous phase containing: a. a crosslinked anionic polyelectrolyte (P) including: —at least one monomer unit derived from 2-methyl-2-[(1-oxo-2-propenyl) amino] 1-propanesulfonic acid in the form of a free or partially or totally salified acid; —at least one monomer unit derived from at least one monomer selected from the elements of the group consisting of acrylamide, N,N-dimethyl acrylamide, methacrylamide, N-isopropyl acrylamide, N-tert-butyl acrylamide; and —at least one monomer unit derived from a polyethylenic crosslinking monomer (AR); b. tetrasodium salt of glutamic acid, N,N diacetic acid.

### CROSS-REFERENCE RELATED TO APPLICATIONS

This application is the U.S. national phase of International Application No. PCT/EP2020/084830 filed Dec. 7, 2020 which designated the U.S. and claims priority to FR Patent Application No. 1913979 filed Dec. 9, 2019, the entire contents of each of which are hereby incorporated by reference.

### BACKGROUND OF THE INVENTION

### Field of the Invention

The present invention relates to a self-invertible inverse latex comprising a novel sequestering agent, to the process for preparing a self-invertible inverse latex, to the use of said self-invertible inverse latexes as thickeners used to prepare cosmetic or pharmaceutical compositions for topical use, and also to said compositions thus prepared.

### Description of the Related Art

Polymers are widely used today in topical cosmetic formulations and represent the second most widely used family of products in formulations of this type. Cosmetic compositions contain polar phases, for instance phases consisting of water, and in most cases require the use of rheology-modifying polymers to increase the viscosity of these polar phases, and also to confer a well-defined rheological behaviour.

Mention may be made, among the rheology-modifying polymers for polar phases, of natural polymers or else of synthetic polymers, and in particular of linear or branched, crosslinked or noncrosslinked anionic or cationic or amphiphilic polyelectrolyte type. These polymers, once introduced into polar phases, have the property of spreading out under the effect of electrostatic repulsions due to the presence of the (negative and/or positive) charges on the linear or branched, noncrosslinked or crosslinked polymer backbone. Rheology-modifying agents provide both an increase in the viscosity of the polar phase, and also a degree of consistency of and/or a stabilizing effect on the cosmetic, dermocosmetic or demopharmaceutical formula use to be thickened.

In order to meet consumer needs and to improve topical cosmetic formulations, scientists have developed new innovative and varied polymer systems. Thus, the polymers used in a topical cosmetic or a dermocosmetic may act as film-forming agents, rheology modifiers, enable the stabilization of the fatty phases in the emulsions (of water-in-oil type or of oil-in-water type) or the stabilization of particles (pigments or fillers), or else confer particular sensory properties after application to the skin (for instance softness to the touch, ease of handling and application, freshness effect, etc.), also having a direct impact on the appearance of the formula (translucent or opaque).

The rheology-modifying polymers for aqueous phases, mainly polyelectrolytes, result from the radical polymerization of (meth)acrylate type monomers, i.e. esters derived from acrylic acid or methacrylic acid, or else derivatives of acrylamide.

Nowadays, these polymers, which may be in the form of an inverse latex, a concentrated inverse latex, or a powder make it possible to meet the customers' needs in terms of thickening performance, in a polar solvent, such as water for example. The aqueous gels obtained once these polymers are dispersed in water have a smooth appearance, free from grains or lumps, with particular sensory properties to the touch, and also an ease of handling and of application.

The liquid form, known under the name “self-invertible inverse latex”, or its concentrated liquid form, is a composition that is in the form of a water-in-oil emulsion and comprises:
an aqueous phase, itself comprising at least one polymer of linear and/or branched and/or crosslinked anionic, or cationic, or ampholytic polyelectrolyte type,
a fatty phase comprising at least one oil,
at least one emulsifying surfactant (S₁) of water-in-oil type,
at least one emulsifying surfactant (S₂) of oil-in-water type, said polymer being obtained by the use of an inverse emulsion radical polymerization process.

Radical polymerization is known for its sensitivity to the presence of impurities, even in small amounts. Compounds which may lead to a decrease in the rate of polymerization at low concentration are known as inhibitors or retarders. However, the distinction between these two effects is not always simple, and the same compound may have both harmful contributions depending on its concentration in the medium or the nature of the monomers and of the reaction medium. Reproducible performance of the thickening polymers for aqueous phases must be guaranteed in order to ensure a consistent quality of the topical cosmetic formulations containing these polymers. For this, the manufacturers must ensure that the polymerization reactions repeatedly follow the same kinetics, more particularly regarding the inhibition time, the reaction exothermicity (° C./min), and the total duration of the polymerization reaction over time. Given these constraints, particular attention is given to the factors that may influence the start of the radical polymerization reaction, for example the presence of oxygen which may retard the polymerization reaction by reacting with the radicals generated. These new peroxide radicals exhibit a lower reactivity, since the initiation capacity is reduced. This results in a lower initiation step and a lower propagation rate, therefore ultimately leading to polymers that have different thickening properties. A step of deoxygenation of the medium, notably by purging with nitrogen before starting the polymerization reaction, thus proves necessary.

Another factor directly impacting the polymerization is the presence of metallic species (Fe²⁺, Fe³⁺, Cu²⁺, . . . ) which, in turn, generate an inhibitory effect. In this case, the inhibition may occur during the initiation phase by the reaction of the initiating radicals with metallic impurities, so that the active radical center then becomes incapable of fixing another monomer unit and becomes inactive during the polymerization.

The metal ions mentioned above may potentially originate from the raw materials or else from the equipment.

The monomers used for the preparation of self-invertible inverse latexes may have traces of metal cations. Similarly, it is not impossible to envisage the presence of metallic contaminants in the industrial equipment receiving the polymerization reactions. In most cases, the equipment is made of stainless steel (commonly called stainless steel), and there are several types of stainless steel which differ according to their composition. Stainless steel is an iron-based alloy added to which are nickel, chromium or molybdenum in certain cases. It is chromium which gives stainless steel its antioxidant properties since in the presence of oxygen it is capable, by itself, of regenerating its surface chromium oxide layer, referred to as a passive layer.

However, it is not impossible that, in prolonged contact with sources of pollution, acids, moisture, spray or iron-laden dust, or in the case of deep scratches, the protective layer will then de-passivate (therefore activate) and the stainless steel will oxidize more quickly than it will be capable of protecting itself. In these cases, the appearance of rust can be found, which rust is therefore a source of iron-based metal contaminants.

Considering the risks associated with the presence of all these sources of metal contaminants, the use of a sequestering agent is essential. The product generally used is the pentasodium salt of diethylenetriaminepentaacetic acid (also known under the brand name Versenex™ 80).

However, the change in the European regulations regarding the classification of the pentasodium salt of diethylenetriaminepentaacetic acid has led to an alternative solution as sequestering agent for the preparation of self-invertible inverse latexes being sought.

From there, a problem which arises is that of providing a new inverse latex with a new sequestering agent that is as efficient as the pentasodium salt of diethylenetriaminepentaacetic acid and having properties which exhibit greater conformity with the changes in regulations.

### SUMMARY OF THE INVENTION

A solution of the present invention is a self-invertible inverse latex comprising an aqueous phase comprising:

a. a crosslinked anionic polyelectrolyte (P) consisting of:
at least one monomer unit derived from 2-methyl-2-[(1-oxo-2-propenyl)amino]-1-propanesulfonic acid in free acid form or partially or totally salified form; and
at least one monomer unit derived from at least one monomer chosen from the elements of the group consisting of acrylamide, N,N-dimethylacrylamide, methacrylamide, N-isopropylacrylamide, and N-(tert-butyl)acrylamide; and
at least one monomer unit derived from a polyethylenic crosslinking monomer (AR);

b. glutamic acid, N,N diacetic acid, tetrasodium salt.

In point of fact, the self-invertible inverse latex according to the invention can exhibit one or more of the following characteristics:
the aqueous phase comprises at least 0.01 mol % of glutamic acid, N,N diacetic acid, tetrasodium salt;
the polyethylenic crosslinking monomer (AR) is chosen from methylenebis(acrylamide), ethylene glycol dimethacrylate, diethylene glycol diacrylate, ethylene glycol diacrylate, diallylurea, triallylamine, trimethylolpropane triacrylate, diallyloxyacetic acid or a salt thereof, such as sodium diallyloxyacetate, or a mixture of these compounds;
the crosslinking monomer (AR) is methylenebis(acrylamide) or triallylamine;
the crosslinked anionic polyelectrolyte comprises:
a proportion of between 20% and 90%, more particularly between 20% and 80%, and even more particularly between 32% and 70% by weight of the monomer unit derived from 2-methyl-2-[(1-oxo-2-propenyl)amino]-1-propanesulfonic acid in free acid form or partially or totally salified form;
A proportion of between 10% and 80%, more particularly between 20% and 80%, and even more particularly between 30% and 68% by weight of the monomer unit derived from at least one monomer chosen from the elements of the group consisting of acrylamide, N,N-dimethylacrylamide, methacrylamide, N-isopropylacrylamide, N-(tert-butyl)acrylamide, and
a proportion of greater than 0 mol % and less than or equal to 1 mol %, more particularly a molar proportion less than or equal to 0.5 mol %, more particularly less than or equal to 0.25 mol % and very particularly less than or equal to 0.1 mol %, and more particularly greater than or equal to 0.005 mol % of monomer units derived from at least one polyethylenic crosslinking monomer (AR).

For the purposes of the present invention, crosslinked anionic polyelectrolyte (P) denotes, for the polymer (P), a nonlinear polyelectrolyte which is provided in the form of a three-dimensional network which is insoluble in water but which can swell in water and which then results in a chemical gel being obtained.

For the purposes of the present invention, the term “salified” indicates that the acid function present in a monomer exists in an anionic form combined in salt form with a cation, in particular alkali metal salts, such as sodium or potassium cations, or such as cations of nitrogenous bases, such as the ammonium salt, the lysine salt or the monoethanolamine salt (HOCH₂—CH₂—NH₃⁺) They are preferably sodium or ammonium salts.

According to one particular aspect of the present invention, said self-invertible inverse latex as defined above comprises from 20% by weight to 90% by weight, and more particularly from 30% by weight to 90% by weight, more particularly from 30% by weight to 80% by weight, and even more particularly from 33% by weight to 80% by weight of said crosslinked anionic polyelectrolyte (P).

According to another particular aspect of the present invention, the molar proportion of monomer units derived from 2-methyl-2-[(1-oxo-2-propenyl)amino]-1-propanesulfonic acid in free acid or partially or totally salified form present in said crosslinked anionic polyelectrolyte (P) is greater than or equal to 32 mol % and less than or equal to 100 mol %, more particularly greater than or equal to 40 mol % and less than or equal to 100 mol %. According to one particular aspect of the present invention, the 2-methyl-2-[(1-oxo-2-propenyl)amino]-1-propanesulfonic acid is in the sodium or ammonium salt form.

A subject of the present invention is also a process for preparing a self-invertible inverse latex as defined above, comprising the following steps:

a) preparing the aqueous phase as defined above,

b) preparing an organic phase comprising at least one oil (O) and an emulsifying surfactant system (S₁) of water-in-oil type,

c) mixing the aqueous phase and the organic phase prepared in steps a) and b) and emulsifying so as to form an emulsion,

d) inerting the emulsion with nitrogen,

e) initiating the polymerization reaction by introducing a free-radical initiator into the inerted emulsion, and

f) introducing into the reaction medium resulting from step e) an emulsifying surfactant system (S₂) of oil-in-water type at a temperature between 30° C. and 60° C.

Depending on the case, the process according to the invention may have one or more of the features below:
in step e) the radical initiator is a redox pair which generates hydrogen sulfite (HSO₃⁻) ions, such as the cumene hydroperoxide/sodium metabisulfite (Na₂S₂O₅) pair or the cumene hydroperoxide/thionyl chloride (SOCl₂) pair;
in step e), a polymerization coinitiator, preferably azobis(isobutyronitrile), is introduced into the inerted emulsion;
in step a), the pH of the aqueous phase is adjusted between 3.0 and 7.0, more particularly between 3.5 and 6.5, more particularly still between 4.0 and 6.5;
the reaction medium derived from step e) is concentrated by distillation before carrying out step f);
the reaction medium derived from step e) or f) is spray-dried;

The term “oil (0)” notably denotes, in the definition of said self-invertible inverse latex:
linear alkanes including from 11 to 19 carbon atoms;
branched alkanes comprising from 7 to 40 carbon atoms, such as isododecane, isopentadecane, isohexadecane, isoheptadecane, isooctadecane, isononadecane or iso eicosane, or mixtures of some of them, such as those mentioned below and identified by their INCI name: C₂₋₈ isoparaffin, C₈₋₉ isoparaffin, C₉₋₁₁ isoparaffin, C₉₋₁₂ isoparaffin, C₉₋₁₃ isoparaffin, C₉₋₁₄ isoparaffin, C₉₋₁₆ isoparaffin, C₁₀₋₁₁ isoparaffin, C₁₀₋₁₂ isoparaffin, C₁₀₋₁₃ isoparaffin, C₁₁₋₁₂ isoparaffin, C₁₁₋₁₃ isoparaffin, C₁₁₋₁₄ isoparaffin, C₁₂₋₁₄ isoparaffin, C₁₂₋₂₀ isoparaffin, C₁₃₋₁₄ isoparaffin, C₁₃₋₁₆ isoparaffin;
cycloalkanes optionally substituted with one or more linear or branched alkyl radicals;
white mineral oils, such as the products sold under the following names: Marcol™ 52, Marcol™ 82, Drakeol™ 6VR, Eolane™ 130, Eolane™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes including from 15 to 19 carbon atoms, said alkanes being linear alkanes, branched alkanes and cycloalkanes, and more particularly the mixture (M₁) which comprises, for 100% of the weight thereof, a proportion by weight of branched alkanes of greater than or equal to 90% and less than or equal to 100%; a proportion by weight of linear alkanes of greater than or equal to 0% and less than or equal to 9%, and more particularly less than 5%, and a proportion by weight of cycloalkanes of greater than or equal to 0% and less than or equal to 1%, for example the mixtures sold under the name Emogreen™ L15 or Emogreen™ L19;
the fatty alcohol ethers of formula (IV):

Z₁—O—Z₂ (IV),

wherein Z₁ and Z₂, which may be identical or different, represent a linear or branched alkyl radical comprising from 5 to 18 carbon atoms, for example dioctyl ether, didecyl ether, didodecyl ether, dodecyl octyl ether, dihexadecyl ether, (1,3-dimethylbutyl) tetradecyl ether, (1,3-dimethylbutyl) hexadecyl ether, bis(1,3-dimethylbutyl) ether or dihexyl ether;
monoesters of fatty acids and of alcohols of formula (V):

R′₁—(C═O)—O—R′₂ (V),

wherein R′₁—(C═O) represents a saturated or unsaturated, linear or branched acyl radical comprising from 8 to 24 carbon atoms, and R′2 represents, independently of R′₁, a saturated or unsaturated, linear or branched hydrocarbon-based chain comprising from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, isostearyl isostearate;
diesters of fatty acids and of glycerol of formula (VI) and of formula (VII):

R′₃—(C═O)—O—CH₂—CH(OH)—CH₂—O—(C═O)—R′₄ (VI)

R′₅—(C═O)—O—CH₂—CH[O—(C═O)—R′₆]—CH₂—OH (VII),

formulae (VI) and (VII) wherein R′₃—(C═O), R′₄—(C═O), R′₅—(C═O) and R′₆—(C═O), which may be identical or different, represent a saturated or unsaturated, linear or branched acyl group including from 8 to 24 carbon atoms;

triesters of fatty acids and of glycerol of formula (VIII):

R′₇—(C═O)—O—CH₂—CH[O—(C═O)—R″₈]—CH₂—O—(C═O)—R″₉ (VIII),

wherein R′₇—(C═O), R′₈—(C═O) and R′₉—(C═O), which may be identical or different, represent a saturated or unsaturated and linear or branched acyl group comprising from 8 to 24 carbon atoms;

According to another particular aspect of the present invention, said oil (O) is chosen from undecane, tridecane, isododecane or isohexadecane, mixtures of alkanes and isoalkanes and cycloalkanes, such as the mixture (M₁) as defined above and the mixtures sold under the name Emogreen™ L15, Emogreen™ L19, Emosmart™ L15, Emosmart™ L19, Emosmart™ V21, Isopar™ L or Isopar™ M; the white mineral oils sold under the name Marcol™ 52, Marcol™ 82, Drakeol™ 6VR, Eolane™ 130 or Eolane™ 150; hemisqualane, squalane, hydrogenated polyisobutene or hydrogenated polydecene; dioctyl ether or didecyl ether; isopropyl myristate, hexyl palmitate, octyl palmitate, isostearyl isostea rate, octanoyl/decanoyl triglyceride, hexadecanoyl/octadecanoyl triglyceride or the triglycerides resulting from rapeseed oil, sunflower oil, linseed oil or palm oil.

In said self-invertible inverse latex which is a subject of the present invention, the emulsifying system (S₁) of water-in-oil type consists either of a single emulsifying surfactant or of a mixture of emulsifying surfactants, provided that said resulting emulsifying surfactant system (S₁) has a sufficiently low HLB value to bring about the formation of emulsions of water-in-oil type.

As emulsifying surfactant (S₁) of water-in-oil type, examples include esters of anhydrohexitol and of linear or branched, saturated or unsaturated aliphatic carboxylic acids, comprising from 12 to 22 carbon atoms, optionally substituted with one or more hydroxyl groups, and more particularly esters of anhydrohexitol chosen from anhydrosorbitols and anhydromannitols and of linear or branched, saturated or unsaturated aliphatic carboxylic acids comprising from 12 to 22 carbon atoms, optionally substituted with one or more hydroxyl groups.

According to another particular aspect of the present invention, said emulsifying surfactant system (S₁) of water-in-oil type is chosen from the elements of the group consisting of sorbitan laurate, for example that sold under the name Montane™ 20, sorbitan palmitate, for example that sold under the name Montane™ 40, sorbitan stearate, for example that sold under the name Montane™ 60, sorbitan oleate, for example that sold under the name Montane™ 80, sorbitan sesquioleate, for example that sold under the name Montane™ 85, sorbitan trioleate, for example that sold under the name Montane™ 83, sorbitan isolaurate, sorbitan isostearate, for example that sold under the name Montane™ 70, mannitan laurate, mannitan oleate and a mixture of these esters; polyesters with a molecular weight of between 1000 and 3000 and resulting from the condensation between a polyisobutenyl succinic acid or its anhydride, such as Hypermer™ 2296, or the mixture sold under the brand name Simaline™ IE 501 A, the polyhydroxystearates of polyglycols of formula (IX):

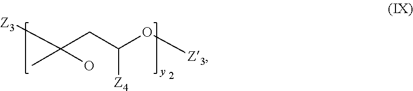

formula (IX) wherein y₂ represents an integer greater than or equal to 2 and less than or equal to 50, Z₄ represents a hydrogen atom, a methyl radical or an ethyl radical, and Z₃ represents a radical of formula (X):

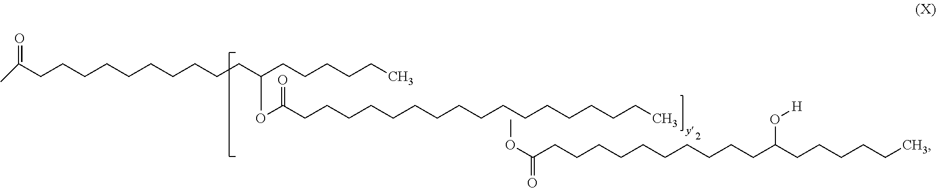

formula (X) wherein y′₂ represents an integer greater than or equal to 0 and less than or equal to 10, more particularly greater than or equal to 1 and less than or equal to 10, and Z′₃ represents a radical of formula (X) as defined above, with Z′₃ being identical to or different than Z₃, or a hydrogen atom.

There is, as example of emulsifying surfactant of water-in-oil type of formula (IX) which can be used to prepare the emulsifying system (S₁), PEG-30

dipolyhydroxystearate sold under the name Simaline™ WO, or else the mixtures comprising PEG-30 dipolyhydroxystearate and sold under the names Simaline™ IE 201 A and Simaline™ IE 201 B, or also the mixture comprising trimethylolpropane-30 tripolyhydroxystearate sold under the name Simaline™ IE 301 B.

According to a particular aspect of the invention, the emulsifying system of oil-in-water type (S₂) comprises, per 100% of its weight, a proportion of greater than or equal to 50% by weight and less than or equal to 100% of a composition (Ce) which comprises, per 100% of its weight:
from 10% by weight to 60% by weight, more particularly from 15% by weight to 60% by weight and very particularly from 15% by weight to 50% by weight of at least one compound of formula (I):

HO—[CH₂—CH(OH)—CH₂—O]ₙ—H (I)

wherein n represents an integer greater than or equal to 1 and less than or equal to 15;
from 40% by weight to 90% by weight, more particularly from 40% by weight to 85% by weight and very particularly from 50% by weight to 85% by weight of at least one compound of formula (II):

R₁—(C═O)—[O—CH₂—CH(OH)—CH₂]ₚ—OH (II),

wherein p, which is different than or identical to n, represents an integer greater than or equal to 1 and less than or equal to 15; and wherein the group R₁—(C═O)— represents a saturated or unsaturated, linear or branched aliphatic radical comprising from 6 to 22 carbon atoms; and optionally
up to 30% by weight, more particularly from 0% by weight to 25% by weight and very particularly from 0% by weight to 20% by weight of at least one composition (C₁₁) represented by the formula (III):

HO—[CH₂—CHOH—CH₂—O-]_{q}-(G)ᵣ—H (III),

wherein q, which is different than or identical to n, represents an integer greater than or equal to 1 and less than or equal to 3, G represents a reducing sugar residue and r represents a decimal number greater than or equal to 1.05 and less than or equal to 5.00,
said composition (C₁₁) consisting of a mixture of the compounds of formulae (III₁), (III₂), (III₃), (III₄) and (III₅):

HO—[CH₂—CHOH—CH₂—O—]_{q}—O-(G)₁-H (III₁),

HO—[CH₂—CHOH—CH₂—O—]_{q}—O-(G)₂-H (III₂),

HO—[CH₂—CHOH—CH₂—O—]_{q}—O-(G)₃-H (III₃),

HO—[CH₂—CHOH—CH₂—O—]_{q}—O-(G)₄-H (III₄),

HO—[CH₂—CHOH—CH₂—O—]_{q}—O-(G)₅-H (III₅),

in molar proportions of said compounds of formulae (III₁), (III₂), (III₃), (III₄) and (III₅) respectively equal to a₁, a₂, a₃, a₄ and as, such that the sum (a₁+a₂+a₃+a₄+a₅) is equal to 1, and such that the sum (a₁+2a₂+3a₃+4a₄+5a₅) is equal to r.

The emulsifying system (S2) of oil-in-water type consists either of the composition (Ce) alone or of a mixture of said composition (Ce) with one or more other emulsifying surfactants, provided that said resulting emulsifying system (S2) has a sufficiently high HLB value to bring about the formation of emulsions of oil-in-water type.

The term “reducing sugar” denotes, in formula (III) as defined above, the saccharide derivatives that do not have, in their structures, any glycoside bond established between an anomeric carbon and the oxygen of an acetal group as defined in the reference publication: “Biochemistry”, Daniel Voet/Judith G. Voet, page 250, John Wiley & Sons, 1990. The oligomeric structure (G)ₓ may be in any isomeric form, whether it is optical isomerism, geometrical isomerism or regioisomerism; it may also represent a mixture of isomers.

Regarding the polymerization reaction, it is initiated in step e) at a preferential temperature of 10° C., then carried out either quasi-adiabatically up to a temperature above or equal to 50° C., or by controlling the temperature.

Another subject of the invention is the use of said self-invertible inverse latex as defined above as thickening and/or emulsifying and/or stabilizing agent for a topical cosmetic or pharmaceutical composition.

Another subject of the invention is a topical cosmetic composition (F) or a topical pharmaceutical composition (G), characterized in that it comprises, as thickening agent, per 100% of its total weight, between 0.1% and 10% by weight of said self-invertible inverse latex as defined above.

The expression “topical” used in the definitions of said compositions (F) and (G) means that they are employed by application to the skin, the hair, the scalp or the mucus membranes, whether it is a direct application, in the case of a cosmetic, dermocosmetic, dermopharmaceutical or pharmaceutical preparation, or an indirect application, for example in the case of a body care product in the form of a textile or paper wipe or of sanitary products intended to be in contact with the skin or the mucus membranes.

Said compositions (F) and (G) are generally provided in the form of an aqueous or aqueous/alcoholic or aqueous/glycol solution, in the form of a suspension, of an emulsion, of a microemulsion or of a nanoemulsion, whether they are of water-in-oil, oil-in-water, water-in-oil-in-water or oil-in-water-in-oil type.

Said compositions (F) and (G) can be packaged in a bottle, in a device of “pump-action spray” type, in pressurized form in an aerosol device, in a device equipped with a perforated wall, such as a grille, or in a device equipped with a ball applicator (known as a “roll-on”).

In general, said compositions (F) and (G) also comprise excipients and/or active principles habitually employed in the field of topical formulations, in particular cosmetic, dermocosmetic, pharmaceutical or dermopharmaceutical formulations, such as thickening and/or gelling surfactants, stabilizers, film-forming compounds, hydrotropic agents, plasticizing agents, emulsifying and coemulsifying agents, opacifying agents, pearlescent agents, superfatting agents, sequestering agents, chelating agents, antioxidants, fragrances, preservatives, conditioning agents, whitening agents intended for bleaching body hairs and the skin, active principles intended to contribute a treating action with regard to the skin or hair, sunscreens, pigments or inorganic fillers, particles providing a visual effect or intended for the encapsulation of active principles, exfoliating particles or texturing agents.

Examples of foaming and/or detergent surfactants which can be combined with said self-invertible inverse latex as defined above in said compositions (F) and (G) include anionic, cationic, amphoteric or nonionic foaming and/or detergent surfactants.

Examples of anionic foaming and/or detergent surfactants which can be combined with said self-invertible inverse latex as defined above in said compositions (F) and (G) include alkali metal, alkaline earth metal, ammonium, amine or aminoalcohol salts of alkyl ether sulfates, of alkyl sulfates, of alkylamido ether sulfates, of alkylaryl polyether sulfates, of monoglycerides sulfates, of α-olefinsulfonates, of paraffinsulfonates, of alkyl phosphates, of alkyl ether phosphates, of alkylsulfonates, of alkylamidesulfonates, of alkylarylsulfonates, of alkylcarboxylates, of alkyl sulfosuccinates, of alkyl ether sulfosuccinates, of alkylamide sulfosuccinates, of alkyl sulfoacetates, of alkylsarcosinates, of acylisethionates, of N-acyltaurates, of acyl lactylates, of N-acylated derivatives of amino acids, of N-acylated derivatives of peptides, of N-acylated derivatives of proteins or of N-acylated derivatives of fatty acids.

The foaming and/or detergent amphoteric surfactants which can be combined with said self-invertible inverse latex as defined above in said compositions (F) and (G) include alkyl betaines, alkyl amido betaines, sultaines, alkyl amidoalkyl sulfobetaines, imidazoline derivatives, phosphobetaines, amphopolyacetates and amphopropionates.

The foaming and/or detergent cationic surfactants which can be combined with said self-invertible inverse latex as defined above in said compositions (F) and (G) particularly include quaternary ammonium derivatives.

The foaming and/or detergent nonionic surfactants which can be combined with said self-invertible inverse latex as defined above in said compositions (F) and (G) more particularly include alkyl polyglycosides comprising a linear or branched and saturated or unsaturated aliphatic radical and comprising from 8 to 16 carbon atoms, such as octyl polyglucoside, decyl polyglucoside, undecylenyl polyglucoside, dodecyl polyglucoside, tetradecyl polyglucoside, hexadecyl polyglucoside or 1,12-dodecanediyl polyglucoside; ethoxylated hydrogenated castor oil derivatives, such as the product sold under the INCI name “PEG-40 hydrogenated castor oil”; polysorbates, such as Polysorbate 20, Polysorbate 40, Polysorbate 60, Polysorbate 70, Polysorbate 80 or Polysorbate 85; coconut amides; or N-alkylamines.

Examples of thickening and/or gelling surfactants which can be combined with said self-invertible inverse latex as defined above in said compositions (F) and (G) include optionally alkoxylated alkyl polyglycoside fatty esters, such as ethoxylated methyl polyglucoside esters, for example the PEG 120 methyl glucose trioleate and the PEG 120 methyl glucose dioleate sold respectively under the names Glucamate™ LT and Glucamate™ DOE-120; alkoxylated fatty esters, such as the PEG 150 pentaerythrityl tetrastearate sold under the name Crothix™ DS53 or the PEG 55 propylene glycol oleate sold under the name Antil™ 141; fatty-chain polyalkylene glycol carbamates, such as the PPG-14 laureth isophoryl dicarbamate sold under the name Elfacos™ T211 or the PPG-14 palmeth-60 hexyl dicarbamate sold under the name Elfacos™ GT2125.

Examples of thickening and/or gelling agents which can be combined with said self-invertible inverse latex as defined above in said compositions (F) and (G) include copolymers of AMPS and of alkyl acrylates, the carbon chain of which comprises between 4 and 30 carbon atoms and more particularly between 10 and 30 carbon atoms, linear, branched or crosslinked terpolymers of at least one monomer bearing a free, partially salified or totally salified strong acid function with at least one neutral monomer and at least one monomer of formula (XIII):

CH₂═C(R′₃)—C(═O)—[CH₂—CH₂—O]n′—R′₄ (XIII)

wherein R′₃ represents a hydrogen atom or a methyl radical, R′₄ represents a linear or branched alkyl radical comprising from 8 to 30 carbon atoms and n′ represents a number greater than or equal to 1 and less than or equal to 50.

Examples of thickening and/or gelling agents which can be combined with said self-invertible inverse latex as defined above in said compositions (F) and (G) include polysaccharides consisting solely of monosaccharides, such as glucans or glucose homopolymers, glucomannoglucans, xyloglycans, galactomannans, the degree of substitution (DS) of the D-galactose units on the main D-mannose chain of which is between 0 and 1 and more particularly between 1 and 0.25, such as galactomannans originating from cassia gum (DS=⅕), locust bean gum (DS=¼), tara gum (DS=⅓), guar gum (DS=½) or fenugreek gum (DS=1).

Examples of thickening and/or gelling agents which can be combined with said self-invertible inverse latex as defined above in said compositions (F) and (G) include polysaccharides consisting of monosaccharide derivatives, such as sulfated galactans and more particularly carrageenans and agar, uronans and more particularly algins, alginates and pectins, heteropolymers of monosaccharides and of uronic acids and more particularly xanthan gum, gellan gum, gum arabic exudates and karaya gum exudates, or glucosaminoglycans.

Examples of thickening and/or gelling agents which can be combined with said self-invertible inverse latex as defined above in said compositions (F) and (G) include cellulose, cellulose derivatives, such as methyl cellulose, ethyl cellulose or hydroxypropyl cellulose, silicates, starch, hydrophilic starch derivatives or polyurethanes.

Examples of stabilizing agents which can be combined with said self-invertible inverse latex as defined above in said compositions (F) and (G) include monocrystalline waxes and more particularly ozokerite, inorganic salts, such as sodium chloride or magnesium chloride, or silicone polymers, such as polysiloxane polyalkyl polyether copolymers.

Examples of solvents which can be combined with said self-invertible inverse latex as defined above in said compositions (F) and (G) include water, organic solvents, such as glycerol, diglycerol, glycerol oligomers, ethylene glycol, propylene glycol, butylene glycol, 1,3-propanediol, 1,2-propanediol, hexylene glycol, diethylene glycol, xylitol, erythritol, sorbitol, water-soluble alcohols, such as ethanol, isopropanol or butanol, or mixtures of water and of said organic solvents.

Examples of thermal or mineral waters which can be combined with said self-invertible inverse latex as defined above in said compositions (F) and (G) include thermal or mineral waters having a mineralization of at least 300 mg/I, in particular Avene water, Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier, water, Rochefort water, Saint Christau water, Les Fumades water and Tercis-les-Bains water.

Examples of hydrotropic agents which can be combined with said self-invertible inverse latex as defined above in said compositions (F) and (G) include xylenesulfonates, cumenesulfonates, hexyl polyglucoside, 2-ethylhexyl polyglucoside and n-heptyl polyglucoside.

Examples of emulsifying surface-active agents which can be combined with said self-invertible inverse latex as defined above in said compositions (F) and (G) include nonionic surfactants, anionic surfactants or cationic surfactants.

Examples of emulsifying nonionic surfactants which can be combined with said self-invertible inverse latex as defined above in said compositions (F) and (G) include esters of fatty acids and of sorbitol, such as the products sold under the names Montane™ 40, Montane™ 60, Montane™ 70, Montane™ 80 and Montane™ 85; compositions comprising glycerol stearate and stearic acid ethoxylated with between 5 mol and 150 mol of ethylene oxide, such as the composition comprising stearic acid ethoxylated with 135 mol of ethylene oxide and glycerol stearate sold under the name Simulsol™ 165; mannitan esters; ethoxylated mannitan esters; sucrose esters; methyl glucoside esters; alkyl polyglycosides including a linear or branched and saturated or unsaturated aliphatic radical and comprising from 14 to 36 carbon atoms, such as tetradecyl polyglucoside, hexyldecyl polyglucoside, octadecyl polyglucoside, hexyldecyl polyxyloside, octadecyl polyxyloside, eicosyl polyglucoside, dodecosyl polyglucoside, 2-octyldodecyl polyxyloside or 12-hydroxystearyl polyglucoside; compositions of linear or branched and saturated or unsaturated fatty alcohols and comprising from 14 to 36 carbon atoms and of alkyl polyglycosides such as described above, for example the compositions sold under the names Montanov™ 68, Montanov™ 14, Montanov™ 82, Montanov™ 202, Montanov™ S, Montanov™ W018, Montanov™ L, Fluidanov™ 20X and Easynov™.

Examples of anionic surfactants which can be combined with said self-invertible inverse latex as defined above in said compositions (F) and (G) include glyceryl stearate citrate, cetearyl sulfate, soaps, such as sodium stearate or triethanolammonium stearate, and N-acylated derivatives of amino acids which are salified, for example stearoyl glutamate.

Examples of emulsifying cationic surfactants which can be be combined with said self-invertible inverse latex as defined above in said compositions (F) and (G) include amine oxides, quaternium-82 and the surfactants described in patent application WO 96/00719 and mainly those, the fatty chain of which comprises at least 16 carbon atoms.

Examples of opacifying and/or pearlescent agents which can be combined with said self-invertible inverse latex as defined above in said compositions (F) and (G) include sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate or fatty alcohols comprising from 12 to 22 carbon atoms.

Examples of texturing agents which can be combined with said self-invertible inverse latex as defined above in said compositions (F) and (G) include N-acylated derivatives of amino acids, such as lauroyl lysine sold under the name Aminohope™ LL, starch octenylsuccinate sold under the name Dryflo™, myristyl polyglucoside sold under the name Montanov™ 14, cellulose fibers, cotton fibers, chitosan fibers, talc, sericite or mica.

Examples of deodorant agents which can be combined with said self-invertible inverse latex as defined above in said compositions (F) and (G) include alkali metal silicates, zinc salts, such as zinc sulfate, zinc gluconate, zinc chloride or zinc lactate; quaternary ammonium salts, such as cetyltrimethylammonium salts or cetylpyridinium salts; glycerol derivatives, such as glycerol caprate, glycerol caprylate or polyglycerol caprate; 1,2-decanediol, 1,3-propanediol; salicylic acid; sodium bicarbonate; cyclodextrins; metallic zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, complexes of aluminum chlorohydrate and of glycol, such as the complex of aluminum chlorohydrate and of propylene glycol, the complex of aluminum dichlorohydrate and of propylene glycol, the complex of aluminum sesquichlorohydrate and of propylene glycol, the complex of aluminum chlorohydrate and of polyethylene glycol, the complex of aluminum dichlorohydrate and of polyethylene glycol, or the complex of aluminum sesquichlorohydrate and of polyethylene glycol.

Examples of oils which can be combined with said self-invertible inverse latex as defined above in said compositions (F) and (G) include mineral oils, such as liquid paraffin, liquid petroleum jelly, isoparaffins or white mineral oils; oils of animal origin, such as squalene or squalane; vegetable oils, such as phytosqualane, sweet almond oil, coconut oil, castor oil, jojoba oil, olive oil, rapeseed oil, peanut oil, sunflower oil, wheat germ oil, corn germ oil, soybean oil, cottonseed oil, alfalfa oil, poppy oil, pumpkinseed oil, evening primrose oil, millet oil, barley oil, rye oil, safflower oil, candlenut oil, passionflower oil, hazelnut oil, palm oil, shea butter, apricot kernel oil, calophyllum oil, sisymbrium oil, avocado oil, calendula oil, oils resulting from flowers or vegetables or ethoxylated vegetable oils; synthetic oils, such as fatty acid esters, for example butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate or isocetyl lanolate, fatty acid monoglycerides, diglycerides and triglycerides, such as glycerol triheptanoate, alkylbenzoates, hydrogenated oils, poly(α-olefins), polyolefins, such as poly(isobutene), synthetic isoalkanes, such as isohexadecane or isododecane, or perfluorinated oils; silicone oils, such as dimethylpolysiloxanes, methylphenylpolysiloxanes, silicones modified by amines, silicones modified by fatty acids, silicones modified by alcohols, silicones modified by alcohols and fatty acids, silicones modified by polyether groups, epoxy-modified silicones, silicones modified by fluorinated groups, cyclic silicones and silicones modified by alkyl groups. In the present application, the term “oils” refers to compounds and/or mixtures of compounds which are water-insoluble, and which have a liquid appearance at a temperature of 25° C.

Examples of waxes which can be combined with said self-invertible inverse latex as defined above in said compositions (F) and (G) include beeswax, carnauba wax, candelilla wax, ouricury wax, Japan wax, cork fiber wax, sugarcane wax, paraffin waxes, lignite waxes, microcrystalline waxes, lanolin wax; ozokerite, polyethylene wax, silicone waxes, vegetable waxes, fatty alcohols and fatty acids which are solid at ambient temperature, or glycerides which are solid at ambient temperature. In the present application, the term “waxes” refers to compounds and/or mixtures of compounds which are water-insoluble, and which have a solid appearance at a temperature of greater than or equal to 45° C.

Examples of active principles which can be combined with said self-invertible inverse latex as defined above in said compositions (F) and (G) include vitamins and their derivatives, in particular their esters, such as retinol (vitamin A) and its esters (for example retinyl palmitate), ascorbic acid (vitamin C) and its esters, sugar derivatives of ascorbic acid (such as ascorbyl glucoside), tocopherol (vitamin E) and its esters (such as tocopheryl acetate), vitamin B3 or B10 (niacinamide and its derivatives); compounds showing a lightening or depigmenting action on the skin, such as w-undecylenoyl phenylalanine sold under the name Sepiwhite™ MSH, Sepicalm™ VG, the glycerol monoester and/or the glycerol diester of w-undecylenoyl phenylalanine, w-undecylenoyl dipeptides, arbutin, kojic acid, hydroquinone; compounds showing a soothing action, in particular Sepicalm™ S, allantoin and bisabolol; antiinflammatory agents; compounds showing moisturizing action, such as urea, hydroxyureas, glycerol, polyglycerols, glycerol glucoside, diglycerol glucoside, polyglyceryl glucosides, xylityl glucoside; polyphenol-rich plant extracts, such as grape extracts, pine extracts, wine extracts or olive extracts; compounds showing a slimming or lipolytic action, such as caffeine or its derivatives, Adiposlim™, Adipoless™, fucoxanthin; N-acylated proteins; N-acylated peptides, such as Matrixyl™; N-acylated amino acids; partial hydrolyzates of N-acylated proteins; amino acids; peptides; total hydrolyzates of proteins; soybean extracts, for example Raffermine™; wheat extracts, for example Tensine™ or Gliadine™; plant extracts, such as tannin-rich plant extracts, isoflavone-rich plant extracts or terpene-rich plant extracts; extracts of freshwater or marine algae; marine plant extracts; marine extracts in general, such as corals; essential waxes; bacterial extracts; ceramides; phospholipids; compounds showing an antimicrobial action or a purifying action, such as Lipacide™ C8G, Lipacide™ UG, Sepicontrol™ A5; Octopirox™ or Sensiva™ SC50; compounds showing an energizing or stimulating property, such as Physiogenyl™, panthenol and its derivatives, such as Sepicap™ MP; antiaging active principles, such as Sepilift™ DPHP, Lipacide™ PVB, Sepivinol™, Sepivital™ Manoliva™, Phyto-Age™, Timecode™; Survicode™; antiphotoaging active principles; active principles which protect the integrity of the dermoepidermal junction; active principles which increase the synthesis of the components of the extracellular matrix, such as collagen, elastins or glycosaminoglycans; active principles which act favorably on chemical cell communication, such as cytokines, or physical cell communication, such as integrins; active principles which create a feeling of “heating” on the skin, such as activators of cutaneous microcirculation (such as nicotinic acid derivatives) or products which create a feeling of “coolness” on the skin (such as menthol and derivatives); active principles which improve cutaneous microcirculation, for example venotonics; draining active principles; active principles having a decongestant purpose, such as Ginkgo biloba, ivy, horse chestnut, bamboo, Ruscus, butcher's broom, Centella asiatica, fucus, rosemary or willow extracts; agents for tanning or browning the skin, for example dihydroxyacetone (DHA), erythrulose, mesotartaric aldehyde, glutaraldehyde, glyceraldehyde, alloxan or ninhydrin, plant extracts, for example extracts of red woods of the genus Pterocarpus and of the genus Baphia, such as Pteropcarpus santalinus, Pterocarpus osun, Pterocarpus soyauxii, Pterocarpus erinaceus, Pterocarpus indicus or Baphia nitida, such as those described in the European patent application EP 0 971 683; agents known for their action in facilitating and/or accelerating tanning and/or browning of human skin, and/or for their action in coloring human skin, for example carotenoids (and more particularly β-carotene and γ-carotene), the product sold under the brand name Carrot Oil (INCI name: Daucus carrota, Helianthus annuus sunflower oil) by Provital, which contains carotenoids, vitamin E and vitamin K; tyrosine and/or derivatives thereof, known for their effect on accelerating the tanning of human skin in combination with exposure to ultraviolet radiation, for example the product sold under the brand name SunTan Accelerator™ by Provital, which contains tyrosine and riboflavins (vitamin B), the tyrosine and tyrosinase complex sold under the brand name Zymo Tan Complex by Zymo Line, the product sold under the brand name MelanoBronze™ (INCI name: Acetyl Tyrosine, Monk's pepper extract (Vitex agnus-castus)) by Mibelle, which contains acetyl tyrosine, the product sold under the brand name Unipertan VEG-24/242/2002 (INCI name: butylene glycol and acetyl tyrosine and hydrolyzed vegetable protein and adenosine triphosphate) by Unipex, the product sold under the brand name Try-Excell™ (INCI name: Oleoyl Tyrosine and Luffa Cylindrica (Seed) Oil and Oleic Acid) by Sederma, which contains extracts of marrow seed (or loofah oil), the product sold under the brand name Actibronze™ (INCI name: hydrolyzed wheat protein and acetyl tyrosine and copper gluconate) by Alban Muller, the product sold under the brand name Tyrostan™ (INCI name: potassium caproyl tyrosine) by Synerga, the product sold under the brand name Tyrosinol (INCI name: sorbitan isostearate, glyceryl oleate, caproyl tyrosine) by Synerga, the product sold under the brand name InstaBronze™ (INCI name: dihydroxyacetone and acetyl tyrosine and copper gluconate) by Alban Muller, the product sold under the brand name Tyrosilane (INCI name: methylsilanol and acetyl tyrosine) by Exymol; peptides known for their melanogenesis-activating effect, for example the product sold under the brand name Bronzing SF Peptide powder (INCI name: Dextran and Octapeptide-5) by Infinitec Activos, the product sold under the brand name Melitane (INCI name: Glycerin and Aqua and Dextran and Acetyl Hexapeptide-1) comprising acetyl hexapeptide-1 known for its α-MSH agonist action, the product sold under the brand name Melatimes Solutions™ (INCI name: Butylene Glycol, Palmitoyl Tripeptide-40) by Lipotec, sugars and sugar derivatives, for example the product sold under the brand name Tanositol™ (INCI name: inositol) by Provital, the product sold under the brand name Thalitan™ (or Phycosaccharide™ AG) by CODIF International (INCI name: aqua and hydrolyzed algin (Laminaria digitata) and magnesium sulfate and manganese sulfate) containing an oligosaccharide of marine origin (guluronic acid and mannuronic acid which are chelated with magnesium and manganese ions), the product sold under the brand name Melactiva™ (INCI name: Maltodextrin, Mucuna pruriens seed extract) by Alban Muller, flavonoid-rich compounds, for instance the product sold under the brand name Biotanning (INCI name: Hydrolyzed citrus Aurantium dulcis fruit extract) by Silab and known to be rich in lemon flavonoids (of the hesperidin type); agents intended for the treatment of head hair and/or body hair, for example agents which protect the melanocytes of the hair follicle, which are intended to protect said melanocytes against cytotoxic agents responsible for the senescence and/or the apoptosis of said melanocytes, such as mimetics of the activity of DOPAchrome tautomerase chosen from those described in the European patent application published under the number EP 1 515 688 A2, synthetic molecules which mimic SOD, for example manganese complexes, antioxidant compounds, for example cyclodextrin derivatives, silica-containing compounds derived from ascorbic acid, lysine pyrrolidonecarboxylate or arginine pyrrolidonecarboxylate, combinations of mono- and diester of cinnamic acid and of vitamin C, and more generally those mentioned in the European patent application published under the number EP 1 515 688 A2.

Examples of antioxidants which can be combined with said self-invertible inverse latex as defined above in said compositions (F) and (G) include EDTA and its salts, citric acid, tartaric acid, oxalic acid, BHA (butylhydroxyanisole), BHT (butylhydroxytoluene), tocopherol derivatives, such as tocopheryl acetate, mixtures of antioxidant compounds, such as Dissolvine GL 47S sold by AkzoNobel under the INCI name: Tetrasodium Glutamate Diacetate.

Examples of sunscreens which can be combined with said self-invertible inverse latex as defined above in said compositions (F) and (G) include all those appearing in the amended Cosmetics Directive 76/768/EEC, Annex VII.

The organic sunscreens which can be combined with said self-invertible inverse latex as defined above in said compositions (F) and (G) include the family of the benzoic acid derivatives, such as para-aminobenzoic acids (PABA), in particular monoglycerol esters of PABA, ethyl esters of N,N-dipropoxy PABA, ethyl esters of N,N-diethoxy PABA, ethyl esters of N,N-dimethyl PABA, methyl esters of N,N-dimethyl PABA or butyl esters of N,N-dimethyl PABA; the family of the anthranilic acid derivatives, such as homomenthyl N-acetylanthranilate; the family of the salicylic acid derivatives, such as amyl salicylate, homomenthyl salicylate, ethylhexyl salicylate, phenyl salicylate, benzyl salicylate or p-isopropylphenyl salicylate; the family of the cinnamic acid derivatives, such as ethylhexyl cinnamate, ethyl 4-isopropylcinnamate, methyl 2,5-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxycinnamate, cyclohexyl p-methoxycinnamate, ethyl α-cyano-β-phenylcinnamate, 2-ethylhexyl α-cyano-β-phenylcinnamate or mono(2-ethylhexanoyl)glyceryl di(para-methoxycinnamate); the family of the benzophenone derivatives, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl 4′-phenylbenzophenone-2,5-dicarboxylate, 2-hydroxy-4-(n-octyloxy)benzophenone, 4-hydroxy-3-carboxybenzophenone; 3-(4′-methylbenzylidene)-d,l-camphor, 3-benzylidene-d,l-camphor, camphor benzalkonium methosulfate; urocanic acid, ethyl urocanate; the family of the sulfonic acid derivatives, such as 2-phenylbenzimidazole-5-sulfonic acid and its salts; the family of the triazine derivatives, such as hydroxyphenyl triazine, ethylhexyloxyhydroxyphenyl-4-methoxyphenyltriazine, 2,4,6-trianilino(p-carbo-2′-ethylhexyl-1′-oxy)-1,3,5-triazine, the 4,4-((6-(((1,1-dimethylethyl)amino)carbonyl)phenyl)amino)-1,3,5-triazine-2,4-diyl diimino) bis-(2-ethylhexyl) ester of benzoic acid, 2-phenyl-5-methylbenzoxazole, 2,2′-hydroxy-5-methylphenylbenzotriazole, 2-(2′-hydroxy-5′-(t-octyl)phenyl)benzotriazole, 2-(2′-hydroxy-5′-methyphenyl)benzotriazole; dibenzalazine; dianisoylmethane, 4-methoxy-4″-t-butylbenzoylmethane; 5-(3,3-dimethyl-2-norbornylidene)-3-pentan-2-one; the family of the diphenylacrylate derivatives, such as 2-ethylhexyl 2-cyano-3,3-diphenyl-2-propenoate or ethyl 2-cyano-3,3-diphenyl-2-propenoate; or the family of the polysiloxanes, such as benzylidene siloxane malonate.

The inorganic sunscreens, also known as “inorganic filters”, which can be combined with said self-invertible inverse latex as defined above in said compositions (F) and (G) include titanium oxides, zinc oxides, cerium oxide, zirconium oxide, yellow, red or black iron oxides, or chromium oxides. These inorganic screening agents may or may not be micronized, may or may not have undergone surface treatments and may optionally be presented in the form of aqueous or oily predispersions.

### DESCRIPTION OF THE PREFERRED EMBODIMENTS

The following example illustrates the invention, without, however, limiting it.

### 1- Examples

### 1.1 Preparation of an Inverse Latex (LI₁) Comprising a Crosslinked Copolymer of the Sodium Salt of 2-Methyl-[(1-Oxo-2-Propenyl)Amino]-1-Propanesulfonic Acid and of Acrylamide Containing Glutamic Acid, N,N-Diacetic Acid, Tetrasodium Salt as Sequestering Agent

The following are charged to a beaker, with stirring:
272 g of a commercial solution containing 55% of sodium salt of 2-methyl-[(1-oxo-2-propenyl)amino]-1-propanesulfonic acid
255 g of a solution containing 50% of acrylamide
115 g of 2-methyl-[(1-oxo-2-propenyl)amino]-1-propanesulfonic acid
0.115 g of methylenebisacrylamide
0.6 g of a commercial solution of glutamic acid, N,N diacetic acid, tetrasodium salt
0.14 g of copper sulfate pentahydrate

The pH of the aqueous phase is adjusted to 6 with a 48% sodium hydroxide solution. The organic phase is prepared at the same time by mixing:
220 grams of isohexadecane
21 grams of Montane™ 80⁽¹⁾

(1): Montane™80 is a sorbitan monooleate, an emulsifying surfactant of water-in-oil type, sold by SEPPIC.

The aqueous phase prepared above is gradually added to the oily phase and then dispersed using an Ultra Turrax™ rotor-stator sold by Ika™.

The emulsion obtained is then transferred to a jacketed reactor, and subjected to nitrogen bubbling in order to remove the oxygen. A solution containing 0.56% by weight of cumene hydroperoxide in 5 ml of isohexadecane is introduced and the emulsion is kept stirring for 5 minutes of homogenization at ambient temperature.

The polymerization reaction is initiated by adding 15 ml of an aqueous solution containing 0.2% of sodium metabisulfite. Once the polymerization reaction has finished, the reaction medium is heated at 85° C. for 1 h and then the whole mixture is cooled to approximately 35° C., then 50 g of Montanox™ 80⁽²⁾ are added to the preparation.

(2): Montanox™80 is a polyoxyethylenated derivative of a sorbitan monooleate, used as a surfactant of oil-in-water type, sold by SEPPIC.

The product obtained is referenced (LI₁) and the results of its evaluations are presented in table 1.

### 1.2 Preparation of an Inverse Latex (LI₂) Comprising a Crosslinked Copolymer of the Sodium Salt of 2-Methyl-[(1-Oxo-2-Propenyl)Amino]-1-Propanesulfonic Acid and of Acrylamide Containing Sodium Diethylenetriaminepentaacetate as Sequestering Agent

The same protocol as 1.1 is implemented, but the 0.6 g of a commercial solution of glutamic acid, N,N diacetic acid, tetrasodium salt are replaced with 0.45 g of a solution of sodium diethylenetriamine pentaacetate sold under the brand name Versenex™ 80.

The Product is referenced (LI2).

TABLE 1

| | | | | | analyses | | |
|---|---|---|---|---|---|---|---|
| | | | | | | Viscosity of | Viscosity of |
| | | | | | | aqueous gel at | aqueous gel at |
| | Sequestering | Polymerization | | | Latex viscosity | 2% by weight, | 3% by weight + |
| | agent | inhibi- | exother- | polymer- | 25° C. (mPa · s) | mPa · s | 0.1% NaCl, mPa · s |
| Test | (amount. in | tion | micity | isation | (Brookfield RVT, | (Brookfield RVT, | (Brookfield RVT, |
| No. | mol ppm)(*) | (min) | (° C.) | time (min) | Spindle 3 Speed 20) | Spindle 6 Speed 5) | Spindle 6 Speed 5) |
| [symbol] | Versenex ™ 80 | 1 | 52.2 | 12 | 2500 | 97 000 | 9100 |
| | 120 ppm | | | | | | |
| [symbol] | Dissolvine ™ | 0 | 51.8 | 13 | 2215 | 91 400 | 11 540 |
| | GLDA 475 | | | | | | |
| | (275 ppm) | | | | | | |

[symbol]

indicates data missing or illegible when filed

### Properties of the Copolymers Obtained in Examples 1.1 and 1.2

In conclusion, the tests for copolymerization of 2-methyl-[(1-oxo-2-propenyl)amino]-1-propanesulfonic acid and of acrylamide show that, in the presence of copper cations, glutamic acid, N,N diacetic acid, tetrasodium salt exhibits an efficiency similar to sodium diethylenetriaminepentaacetate.

In each of the tests, the polymerization exhibits similar characteristics: duration of inhibition, of polymerization and exothermicity. The self invertible inverse latexes obtained under these conditions have equivalent thickening properties in water and in the presence of electrolytes.

1. A self-invertible inverse latex comprising an aqueous phase comprising:
a. a crosslinked anionic polyelectrolyte (P) consisting of:
at least one monomer unit derived from 2-methyl-2-[(1-oxo-2-propenyl)amino]-1-propanesulfonic acid in free acid form or partially or totally salified form;
at least one monomer unit derived from at least one monomer chosen from the elements of the group consisting of acrylamide, N,N-dimethylacrylamide, methacrylamide, N-isopropylacrylamide, and N-(tert-butyl)acrylamide; and
at least one monomer unit derived from a polyethylenic crosslinking monomer (AR);
b. glutamic acid, N,N diacetic acid, tetrasodium salt.

2. The inverse latex as claimed in claim 1, wherein the aqueous phase comprises at least 0.01 mol % of glutamic acid, N,N-diacetic acid, tetrasodium salt.

3. The inverse latex as claimed in claim 1, wherein the polyethylenic crosslinking monomer (AR) is chosen from methylenebis(acrylamide), ethylene glycol dimethacrylate, diethylene glycol diacrylate, ethylene glycol diacrylate, diallylurea, triallylamine, trimethylolpropane triacrylate, diallyloxyacetic acid or a salt thereof, or a mixture of these compounds.

4. The inverse latex as claimed in claim 1, wherein the crosslinking monomer (AR) is methylenebis(acrylamide) or triallylamine.

5. The inverse latex as claimed in claim 1, wherein the crosslinked anionic polyelectrolyte comprises, for 100 mol %:
a proportion between 20% and 90% of the monomer unit derived from 2-methyl-2-[(1-oxo-2-propenyl)amino]-1-propanesulfonic acid in free acid form or partially or totally salified form;
a proportion of between 10% and 80% of the monomer unit derived from at least one monomer chosen from the elements of the group consisting of acrylamide, N,N-dimethylacrylamide, methacrylamide, N-isopropylacrylamide, and N-(tert-butyl)acrylamide, and
a proportion of greater than 0 mol % and less than or equal to 1 mol % of monomer units derived from at least one polyethylenic crosslinking monomer (AR).

6. A process for preparing an inverse latex as defined in claim 1, comprising the following steps:
a) preparing the aqueous phase as defined in claim 1,
b) preparing an organic phase comprising at least one oil and an emulsifying surfactant system (S₁) of water-in-oil type,
c) mixing the aqueous phase and the organic phase prepared in steps a) and b) and emulsifying so as to form an emulsion,
d) inerting the emulsion with nitrogen,
e) initiating the polymerization reaction by introducing a free-radical initiator into the inerted emulsion, and
f) introducing into the reaction medium resulting from step e) an emulsifying surfactant system (S₂) of oil-in-water type at a temperature between 30° C. and 60° C.

7. Process as claimed in claim 6, wherein, in step e), the radical initiator is a redox pair which generates hydrogen sulfite (HSO₃⁻) ions.

8. Process according to claim 6, wherein, in step e), a polymerization coinitiator is introduced into the inerted emulsion.

9. Process according to claim 6, wherein, in step a), the pH of the aqueous phase is adjusted between 3.0 and 7.0.

10. Process according to claim 6, wherein the reaction medium derived from step e) is concentrated by distillation before carrying out step f).

11. Process according to claim 6, wherein the reaction medium derived from step e) or f) is spray-dried.

12. A thickening and/or emulsifying and/or stabilizing agent for a topical cosmetic composition, comprising the inverse latex of claim 1.

13. A topical cosmetic composition (F) comprising, as thickening agent, per 100% of its total weight, between 0.1% and 10% by weight of said inverse latex as defined in claim 1.

14. A topical pharmaceutical composition (G) comprising, as thickening agent, per 100% of its total weight, between 0.1% and 10% by weight of said inverse latex as defined in claim 1.

15. The process of claim 7, wherein the redox pair is the cumene hydroperoxide/sodium metabisulfite (Na₂S₂O₅) pair.

16. The process of claim 7, wherein the redox pair is the cumene hydroperoxide/thionyl chloride (SOCl₂) pair.

17. The process of claim 8, wherein the polymerization coinitiator is azobis(isobutyronitrile).

18. The inverse latex as claimed in claim 2, wherein the polyethylenic crosslinking monomer (AR) is chosen from methylenebis(acrylamide), ethylene glycol dimethacrylate, diethylene glycol diacrylate, ethylene glycol diacrylate, diallylurea, triallylamine, trimethylolpropane triacrylate, diallyloxyacetic acid or a salt thereof, or a mixture of these compounds.

19. The inverse latex as claimed in claim 2, wherein the crosslinking monomer (AR) is methylenebis(acrylamide) or triallylamine.

20. The inverse latex as claimed in claim 3, wherein the crosslinking monomer (AR) is methylenebis(acrylamide) or triallylamine.
